(19) United States
(12) Patent Application Publication
(10) Pub. No.: US 2021/0241648 A1
(43) Pub. Date: Aug. 5, 2021

(54) SYSTEMS AND METHODS TO PROVIDE MENTAL DISTRESS THERAPY THROUGH SUBJECT INTERACTION WITH AN INTERACTIVE SPACE

(71) Applicant: Workplace Technologies Research, Inc., San Diego, CA (US)

(72) Inventors: David M. Lehmann, San Diego, CA (US); Lia A. DiBello, San Diego, CA (US)

(21) Appl. No.: 16/776,852

(22) Filed: Jan. 30, 2020

(51) Int. Cl. G09B 19/00; G06F 3/0482; A61B 5/16

(52) U.S. Cl. CPC G09B 19/00; A61B 5/165; G06F 3/0482

(57) ABSTRACT

Systems and methods to provide mental distress therapy through subject interaction with an interactive space are described herein. Exemplary implementations may: store space information and therapy information; effectuate presentation of a user interface on a computing platform associated with a therapist user, the user interface being configured to obtain entry and/or selection of subject condition information and data collection information; generate the interactive space including virtual content associated with the subject condition information; generate behavior logging information reflecting subject interaction with the interactive space based on the data collection information; and/or perform other operations.

### FIELD OF THE DISCLOSURE

The present disclosure relates to systems and methods to provide mental distress therapy through user interaction with an interactive space.

### BACKGROUND

DSM-5 category mental distress disorders, and especially Post-traumatic stress disorder (“PTSD”), are currently considered to be disorders of emotional learning in which fear is learned. A key feature of PTSD is the generalization of the fear cues from the specific event to encompass sensory stimuli, events, and experiences distal to the original trauma. Across the range of traumas, those with PTSD often come to fear bridges and tunnels, crowed locations (e.g., supermarkets, movie theaters, etc.), noisy locations (sports venues, restaurants, etc.), and/or watching the news where other traumas may be reported. Cues to fear have generalized outward causing disabling emotional distress, physiological arousal and avoidant behaviors.

When a trauma occurs, the participant learns to develop a fear response to situations. Previous neutral sensory stimuli embedded in the fabric of the trauma memory become cues to fear. A mechanism underling the type of learning that occurs during fear acquisition is the classical conditioning process.

Current thinking in PTSD treatment regards the trauma as the focal point of exposure therapy, which is widely regarded as the first-line treatment for PTSD. A prototypical treatment session involves the subject recounting their trauma story, in the first person, as if it was happening again. The central idea is to extinguish the fear associated with the trauma by repeatedly narrating it, in a safe environment, until the cues to fear extinguish. However, the fear generated by any given index trauma generalizes well beyond its sensory and experiential bounds. As the fear generalizes to distal cues, it becomes more disabling as PTSD subjects circumscribe their lives to avoid their fear and its associated physiological manifestations.

### SUMMARY

To address the disabling fear and anxiety generated by one or more common distal cues, one or more aspects of the disclosure relates to a system configured to provide mental distress therapy through user interaction with an interactive space. The system may facilitate a treatment approach to PTSD (and/or other mental distress disorders) that may emphasize the cues to fear that are shared across traumas. Subjects may focus on systematic exposure to feared environments distal to the original trauma, which may cause functional impairment (e.g., driving in traffic, going to the grocery store, and/or others). The system may be used to treat individuals with acquired mental health conditions and/or other conditions which can be improved through learning. These include, but are not limited to depressive disorders, anxiety disorders and trauma and stressor related disorders.

One or more implementations of the system may provide platform that mental health therapists may use to build immersive interactive spaces for subjects to interact with during the course of mental distress treatment. Biometric, behavioral, and/or other information about the subject may be collected during the interaction and/or beyond. The collected data may be stored, compiled, and/or analyzed real time to assist the therapist with managing a treatment plan. The interactive space may be designed to engage the user in challenging activities of increasing difficulty as the user gains confidence and skill in their need areas. An easy to use user interface may guide the therapist user into the assembly of scenarios, dashboards to giving feedback, and/or protocols for collecting information about the subject's interaction. Virtual activities have been shown to make it easier and appealing for people to try activities that are threatening, or anxiety provoking in real life, and gradually overcome their fear and anxiety. Although, the same research indicates there are not many easy-to-use products for this purpose, until now. Further, existing solutions solely involving virtual reality, which include wearing a head-mounted display, may not be practical for application in mental distress treatment, as the head-mounted display is required and often cumbersome and wearing the display may invoke feelings of claustrophobia and/or other discomfort.

One or more features and/or functionality of the technology described herein has proven effective for other types of cognitive learning through cognitive reorganization. Accordingly, those skilled in the art may recognize that changes may be made where appropriate to implement the system in a manner which addresses some type of cognitive learning regime other than mental distress therapy. Further, is noted that while one or more features and/or functions described herein are directed to a single interaction with a virtual environment by a subject, this is for illustrative purposes only. Instead, it is to be understood that one or more features and/or functions described herein may be extended to treatment plans which may involve multiple iterations of subject access to a virtual environment. The virtual environment may change through one or more of the iterations. The virtual environment may consistently remain the same through one or more of the iterations. The virtual environment may change and/or remain the same based on subject interaction with one or more of the iterations. In some implementations, iterative application of a virtual environment in the subject therapy plan may facilitate “cognitive reorganization.” Cognitive reorganization through the system may refer to the subjects use of the immersive learning environment provided by the system, where iterative challenges, feedback, and/or rehearsals may dis-equilibrate biases and/or constraints lacked in mental models not aligned with therapy goals in the adaptive unconscious. Subsequently there may exist opening for new knowledge, through iterative success and self-learning from feedback provided by the system. The system may be used for constructing and habituating new mental models in the subjects, that pull the achieve to achieve the goal. The system may achieve results which, historically, were only carried out face to face in exercises between therapist and subject.

The cognitive reorganization may be based on the iterative experience and immediate embedded feedback on the user's response to experiences. The feedback may be granular and/or low density (e.g., color-based response indicators), and/or may be provided by more detailed feedback (e.g., an explicit prompt). Having embedded feedback along a journey through a virtual environment along with explicit objectives have been shown to reorganize a user's default “theory” or interpretation of the situation, e.g., if the feedback is provided when the situation is being interpreted with an inappropriate or inaccurate model of the situation or scene and the user's actions indicate that. The subject may never actually be “instructed” but rather has to discover the correct interpretation of the situation through trial and error. Likewise, the subjects behavior is used as an indicator of his or her interpretation. Eventually, the result of subject interaction may be the development of a mental model of a situation that incorporates other's views, is less egoistic, and/or which takes into consideration all the information available and is generally more adaptive.

One or more implementations of a system configured to provide mental distress therapy through user interaction with an interactive space may include one or more of one or more servers, one or more computing platforms, non-transitory electronic storage, and/or other components. The one or more servers may include one or more physical processors. The one or more servers may communicate with one or more computing platforms via client/server architecture, and/or other communication schemes. The one or more physical processors may be configured by machine-readable instructions. Executing the machine-readable instructions may cause the one or more physical processor to facilitate providing mental distress therapy through user interaction with an interactive space.

The non-transitory electronic storage may be configured to store one or more of space information, therapy information, and/or other information. The space information may define virtual content of an interactive space. The therapy information may specify associations between the virtual content and one or more mental distress conditions. By way of non-limiting illustration, the space information may define first virtual content and/or other virtual content. The therapy information may specify an association between the first virtual content and a first mental distress condition, and/or other associations.

The processor(s) may be configured to effectuate presentation of a user interface on a computing platform associated with a therapist user. The configuration may be determined by a therapist, the client themselves or smart AI type platform. For descriptive purpose, this entity will be described as a therapist. The user interface being configured to obtain entry and/or selection of one or more of subject condition information, data collection information, and/or other information. The subject condition information may include individual mental distress conditions of a subject of the mental distress therapy, and/or other information. The data collection information may include information to be collected about subject interaction with the interactive space during the mental distress therapy.

The processor(s) may be configured to generate the interactive space including the virtual content associated with entered and/or selected ones of the individual mental distress conditions of the subject. The subject interaction with the interactive space may cause the subject to encounter the virtual content associated with the entered and/or selected ones of the individual mental distress conditions. By way of non-limiting illustration, the interactive space may be generated to include the first virtual content based on an entry and/or selection of the first mental distress condition.

The processor(s) may be configured to generate behavior logging information reflecting the subject interaction with the interactive space based on the data collection information and/or other information.

The processor(s) may be configured to parse the behavior logging information into a structured data format. The therapist user may access the structured data format of the behavior logging information to assist the therapist with managing a treatment plan. In some implementations, the processor(s) may be configured to generate an interaction report based on the behavior logging information and/or other information.

As used herein, any association (or relation, or reflection, or indication, or correspondence) involving servers, processors, client computing platforms, and/or another entity or object that interacts with any part of the system and/or plays a part in the operation of the system, may be a one-to-one association, a one-to-many association, a many-to-one association, and/or a many-to-many association or N-to-M association (note that N and M may be different numbers greater than 1).

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured to provide mental distress therapy through subject interaction with an interactive space, in accordance with one or more implementation.

FIG. 2 illustrates a method to provide mental distress therapy through subject interaction with an interactive space, in accordance with one or more implementations.

FIG. 3 illustrates an example of a user interface.

FIG. 4 illustrates an example of a user interface.

FIG. 5 illustrates an example of a user interface.

FIG. 6 illustrates an example of a user interface.

### DETAILED DESCRIPTION

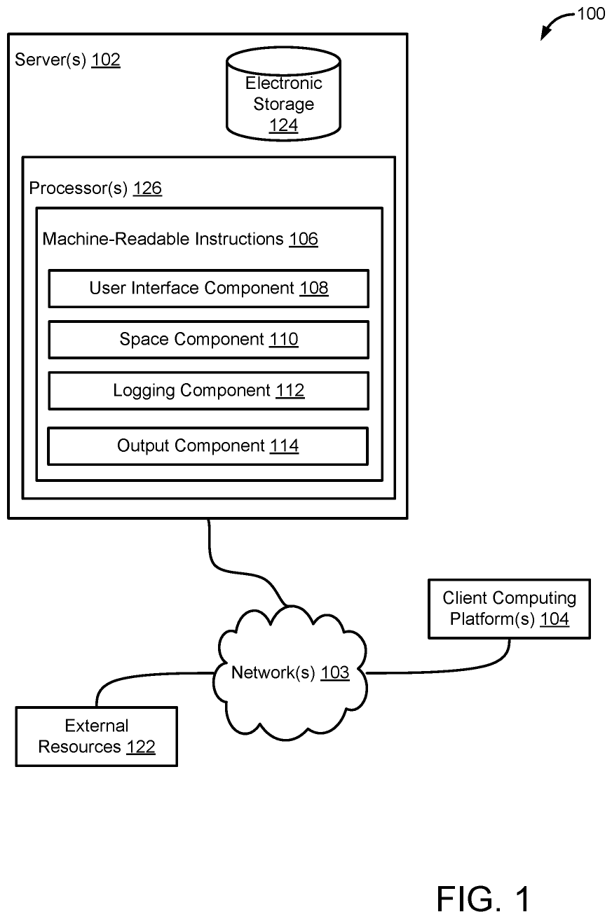

FIG. 1 illustrates a system 100 configured to provide mental distress therapy through user interaction with an interactive space, in accordance with one or more implementations. The system 100 may be configured to provide an engaging, fun, challenging, puzzling, and/or otherwise beneficial virtual experience for the subject that is part of everyday life, but which may include scenes and/or challenges that trigger symptoms of mental distress. The therapist, as a user of the system 100, may design interactive spaces to be less threatening at first. The subject, as a user of the system 100, may experience the interactive space at their own pace, gradually learning to see them as non-threatening and focusing on the positive aspects. The system 100 may be configured to be paused at any time if the subject wants to break and come back later. There may not be time limits and/or other limits to what can be explored. The subject be able to tour the interactive space with limitless experiences. At the same time, the therapist may have the ability to both design the journey through the interactive space with the subject and see how the subject is doing. The system 100 may be configured for behavior tracking and/or recording of decisions and/or responses, and create a chronology for the therapist and the subject for review. One or more features of the system 100 may be configured by one or more of the therapist, the subject, and/or an artificial intelligence system.

An interactive space may include one or more of a virtual environment, an augmented reality (AR) environment, a virtual reality (VR) environment, and/or other interactive spaces. A virtual environment may comprise a simulated space including virtual content. Virtual environments may sometimes be referred to as “virtual worlds.” An augmented reality environment may include views of images of virtual content within a virtual environment superimposed over views of a real-world environment. In some implementations, a user may actively view the real-world environment, for example, through a visor. In some implementations, a user may passively view the real-world environment, for example, through a display that presents images of the real-world environment. A virtual reality environment may include views of a virtual environment. The terms “space” and “environment” in an interactive space may be used interchangeably herein.

There are a number of advantages to the real-time behavioral tracking. Today mentally distressed subjects, such as those suffering PTSD, may not receive help during their normal day when they first sense trouble. One or more implementations of the system may make it possible for therapist to direct therapies that are available to the subject 24/7. Therapists traditionally have limited insights into the subject experiences. The therapists rely upon self-reported from the subject at the beginning of a counseling session. One or more implementations of the system 100 may maintain an account of the subject's activities so the therapist can access this information at any time. The therapist could modify the therapy and/or could make a more fact-based therapy plan. The collected information may be manipulated with analysis including artificial intelligence methods to recognize patterns that might otherwise not be evident to the therapist. The therapist, through an internet of things portal, may access a full accounting of the subject's interactions with the therapeutic protocols. Approaches to therapy include one or more of cognitive behavioral therapy, exposure therapy, desensitization therapy, and/or other approaches. Individual and/or combinations of therapies may be delivered through an interactive space.

In some implementations, system 100 may include one or more of one or more servers 102, one or more client computing platforms 104, external resources 122, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via individual ones of the client computing platform(s) 104. The client computing platform(s) 104 through which subjects access the system 100 may include mobile computing platforms. Mobile computing platforms may include one or more of a smartphone, a laptop, a tablet computer, and/or other computing platform. The client computing platform(s) 104 through which therapists (also referred to herein as “therapist users”) access the system 100 may include mobile computing platforms and/or stationary computing platforms. Stationary computing platforms may include a desktop computer and/or other computing platforms.

Server(s) 102 may be configured by machine-readable instructions 106. Machine-readable instructions 106 may include one or more instruction components. Executing the machine-readable instructions 106 may cause server(s) 102 to facilitate providing mental distress therapy through subject interaction with an interactive space. The instruction components may include computer program components. The instruction components may include one or more of a user interface component 108, a space component 110, a logging component 112, an output component 114, and/or other instruction components.

Server(s) 102 may non-transitory electronic storage 124. The non-transitory electronic storage 124 may be configured to store one or more of space information, therapy information, behavior logging information, subject condition information, data collection information, and/or other information. The space information may define virtual content of an interactive space (see, e.g., space component 110). By way of non-limiting illustration, the space information may define first virtual content and/or other virtual content.

The therapy information may specify associations between the virtual content and one or more mental distress conditions. An association may mean that virtual content includes content and/or content features which may be considered as addressing, identifying, and/or otherwise treating an associated mental distress condition. An association may mean that virtual content includes content and/or content features which may be considered as addressing, identifying, and/or otherwise treating an associated mental distress condition by way of one or more of cognitive behavioral therapy, exposure therapy, desensitization therapy, and/or other approaches which may be conventional in the practice of therapy, may be emerging in the practice of therapy, and/or may be new and/or otherwise unknown in the practice of therapy. In some implementations, sets of virtual content may be associated with one or more mental distress conditions. A mental distress condition may be associated with more than one set. A set of virtual content, described more herein with respect to space component 110, may include combinations of virtual content of an interactive space which may define a journey through the interactive space. By way of non-limiting illustration, the therapy information may specify an association between the first virtual content and a first mental distress condition and/or other mental distress conditions, and/or other associations. The first virtual content may be part of a first set of virtual content associated with the first mental distress condition and/or other mental distress conditions.

The user interface component 108 may be configured to effectuate presentation of a user interface, manually or automatically generated by system software, on a computing platform associated with a therapist user. The user interface being configured to obtain entry and/or selection of one or more of subject condition information, data collection information, and/or other information.

An instance of a user interface may include one or more user interface portions. By way of non-limiting illustration, a user interface may include one or more of an input portion, a display portion, and/or other portions. Individual portions may include one or more user interface elements configured to facilitate user interaction with the user interface. By way of non-limiting illustration, user interface elements may include one or more of text input fields, drop down menus, check boxes, display windows, virtual buttons, and/or other elements configured to facilitate user interaction.

The subject condition information may include individual mental distress conditions of a subject of the mental distress therapy, and/or other information. Mental distress conditions may be specified at one or more levels of granularity. Levels of granularity may include one or more of high level, subject-specific, and/or other specifications. High level may specify a condition, for example, as it may be generally known. Subject-specific may specify the high level and/or include details of condition(s) and/or causes thereof particular to a subject. By way of non-limiting illustration, high level conditions may include one or more of post-traumatic stress disorder (PTSD), overeating, depression, trauma, anxiety, bi-polar, high temper, obsessive compulsive, claustrophobia, and/or other conditions. By way of non-limiting illustration, a subject-specific condition may include PTSD when in large groups of people. By way of non-limiting illustration, a subject-specific condition may include depression during winter. By way of non-limiting illustration, a subject-specific condition may include obsessive compulsive with respect to cleanliness. It is noted that the above descriptions of mental distress conditions are for illustrative purpose only. Instead, it is to be understood that mental distress conditions may include other condition not listed, and/or may be specified at one or more other levels of granularity. Indeed, those skilled in the art may recognize that mental distress may vary widely between subjects, may be described in other ways, and/or may stem from other causes.

The data collection information may include information to be collected about subject interaction with the interactive space during the mental distress therapy. The information to be collected about subject interaction with the interactive space during the mental distress therapy may refer to specific virtual content, specific interactions, and/or other aspects of subject interaction. By way of non-limiting illustration, virtual content may include one or more objectives comprising purposes or goals that the subject interaction with the interactive space is intended to attain or accomplish. Virtual content and subject interaction will be described in more detail herein with respect to space component 110. In some implementations, the data collection information may specify one or more of a start time of the subject interaction, an end time of the subject interaction, identification of one or more objectives, scenes of interest, and/or virtual content, one or more aspects of subject interaction with the interactive space toward attaining or accomplishing the identified ones of the one or more objectives and/or engaging in the scenes, and/or other information.

The user interface component 108 may be configured to effectuate presentation, on the user interface and in response to the entry and/or selection of the subject condition information, individual sets of virtual content associated with the individual mental distress conditions. Individual virtual content in the individual sets of virtual content and/or the individual sets themselves may be configured to be selected by the therapist user for inclusion in the interactive space. By way of non-limiting illustration, user interface component 108 may be configured to, responsive to the entry and/or selection of the first mental distress condition, effectuate presentation of the first set of virtual content associated with the first mental distress condition.

In some implementations, the user interface may facilitate modifying and/or customizing the virtual content and/or sets of virtual content. In some implementations, modifying and/or customizing may be performed at one or more levels of granularity. In some implementations, the user interface may be specifically adapted for use by therapists who may not have technical abilities in the field of virtual environment creation and/or digital animation. Technical abilities in the field of virtual environment creation and/or digital animation may refer to the creation of virtual object through designing of meshes, textures, and/or colors, coding in the virtual object abilities, behaviors, and/or capabilities in the environment, and/or other requirements associated with virtual environment creation and/or digital animation. Instead, in some implementations, modifying and/or customizing may be facilitated through more user-friendly technique such as the selection or deselection of virtual content for inclusion or removal (e.g., via check boxes, drag and drop input, and/or other techniques), while the virtual content itself may be known and/or predefined (e.g., within the space information). However, it is noted that the user interface may allow for more technical manner of costuming and/or modify, e.g., through access to source code defining the virtual content and/or to sophisticated animation software.

Treatment-based templates may be included for novice users who wish to have guidance creating their treatment protocols. Some of these may meet insurance reimburse requirements and/or other requirements. The user interface may have a structure that allows the therapist user to construct therapeutic protocols.

The space component 110 may be configured to generate an interactive space. The space component 110 may be configured to generate an interactive space including the virtual content associated with entered and/or selected ones of the individual mental distress conditions of the subject, and/or other virtual content. In some implementations, generating the interactive space to include the virtual content associated with entered and/or selected ones of the individual mental distress conditions of the subject may include an automated selection of the virtual content associated with entered and/or selected ones of the individual mental distress conditions. For example, virtual content associated with entered and/or selected ones of the individual mental distress conditions of the subject may be identified based on the space information and/or therapy information and automatically selected in response to the entry and/or selection of the subject condition information. The subject condition information may be matched with the therapy information to identify virtual content. By way of non-limiting illustration, the interactive space may be generated to include the first virtual content based on an entry and/or selection of the first mental distress condition.

In some implementations, the interactive space may include the virtual content associated with entered and/or selected ones of the individual mental distress conditions of the subject by virtue of entry and/or selection of individual virtual content and/or individual sets of virtual content specifically selected by a therapist user. By way of non-limiting illustration, virtual content in the individual sets of virtual content and/or the individual sets themselves entered and/or selected by the therapist user for inclusion in the interactive space may comprise the virtual content included in the interactive space. By way of non-limiting illustration, the interactive space may be generated to include the first virtual content based on an entry and/or selection of the first virtual content from the first set of virtual content presented to a therapist user.

The space component 110 may be configured to effectuate presentation of an interactive space on individual client computing platforms of subjects to facilitate subject interaction with the interactive space. In some implementations, the subject interaction with the interactive space may cause the subject to encounter the virtual content associated with the entered and/or selected ones of the individual mental distress conditions and/or encounter other aspects of the interactive space.

An instance a virtual environment of an interactive space may comprise a simulated space that is accessible by users via clients that present the views of the virtual environment. The views may be determined based on a user-perspective. The user perspective may include one or more of first-person, third-person, side scrolling, and/or other perspectives. The simulated space may have a topography, express ongoing real-time interaction by one or more users, and/or include one or more virtual objects positioned within the topography that are capable of locomotion within the topography. In some instances, the topography may be a 2-dimensional topography. In other instances, the topography may be a 3-dimensional topography. The topography may include dimensions of the space and/or surface features of a surface or objects that are “native” to the space. In some instances, the topography may describe a surface (e.g., a ground surface) that runs through at least a substantial portion of the space. In some instances, the topography may describe a volume with one or more bodies positioned therein (e.g., a simulation of gravity-deprived space with one or more celestial bodies positioned therein). An instance executed by the computer components may be synchronous, asynchronous, and/or semi-synchronous.

Within the instance(s) of a virtual environment, users may control virtual objects, simulated physical phenomena (e.g., wind, rain, earthquakes, and/or other phenomena), and/or other elements within the interactive space to interact with the virtual environment, other virtual objects, and/or other users. The virtual objects may include virtual entities such as avatars. As used herein, the term virtual entity may refer to a virtual object present in the interactive space that represents an individual user. A virtual entity may be controlled by the user with which it is associated.

The subject-controlled element(s) may move through and interact with the interactive space (e.g., non-subject characters in the virtual environment and/or other objects in the interactive space). The subject-controlled elements controlled by and/or associated with a given user may be created and/or customized by the given user. The user may have an “inventory” of virtual items and/or currency that the user can use (e.g., by manipulation of a virtual entity or other subject-controlled element, and/or other items) within the interactive space.

Control by users may be exercised through control inputs and/or commands input by the users into a client computing platform. The users may interact with each other, with non-user characters, and/or other entities through communications exchanged within the virtual environment. Such communications may include one or more of textual chat, instant messages, private messages, voice communications, and/or other communications. Communications may be received and entered by the users. Communications may be routed to and from the appropriate users through one or more physical processors 126 and/or through communications which are external to the system 100 (e.g., text messaging services).

The instance of the virtual environment may be persistent. That is, the virtual environment may continue on whether or not individual users are currently logged in and/or participating in the interactive space. A user who logs out of the interactive space and then logs back in some time later may find the virtual environment has been changed through the interactions of other users with the virtual environment during the time the user was logged out. These changes may include changes to the simulated physical space, changes in the user's inventory, changes in other users' inventories, changes experienced by non-subject characters (also referred to as non-player characters, or NPCs), changes to the virtual items available for use in the interactive space, and/or other changes.

The virtual content may include one or more of virtual objects, one or more scenes, one or more objectives, and/or other virtual content. Virtual objects may include virtual items, virtual goods, non-subject controlled virtual entities, and/or other virtual objects. Virtual items and/or goods may represent real-world items and/or goods, fantasy items and/or goods, and/or other content. An objective may comprise a purpose or goal that the subject interaction with the interactive space is intended to attain or accomplish. An objective may be set forth in an interactive space by virtue of one or more of the programming of NPCs (their behaviors, dialogue, and/or other programmable features), placement of virtual objects, the programming of virtual objects (e.g., their behaviors, manners in which the subject may interact with them, and/or other features), and/or other techniques.

In some implementations, the subject interaction with the interactive space to attain or accomplish the one or more objectives may include one or more of control of a subject-controlled virtual entity traversing through a topography of the interactive space, interaction with one or more virtual items, interaction with one or more non-user controlled virtual entities, and/or other interactions. A scene may refer to a setting and/or overall theme of the virtual environment. A setting and/or theme may comprise a unifying subject of the virtual environment including a topography and/or other virtual objects indicative of the unifying subject. By way of non-limiting illustration, a scene may include one or more of a grocery store, a parking lot, a dinner table, a checkout counter, an office building, an outdoor environment, and/or other scenes. It is noted that the above descriptions of scenes are for illustrative purposes only and not to be considered limiting. Instead, it is to be understood that a scene may take on a variety of forms as needed to provide a specific therapy to a subject.

The space component 110 may be configured to effectuate presentation of one or more response indicators in the interactive space based on subject interaction with the interactive space. An individual response indicator may convey feedback of the subject interaction. An individual response indicator may be presented in real time and may convey real-time feedback of the subject interaction. In some implementations, the feedback may be related to a subject's progress in attaining or accomplishing one or more objectives. The feedback may convey one or more of whether the subject is progressing toward attaining or accomplishing one or more objectives, whether the subject is falling behind attaining or accomplishing one or more objectives, whether the subject is stagnant with respect to attaining or accomplishing one or more objectives, and/or other information. In some implementations, a response indicator may comprise one or more of qualitative feedback, quantitative feedback, and/or other information.

In some implementations, qualitative feedback may be provided through presentation of one or more colors within a response indicator, one or more explicit prompts within a response indicator, and/or other information. A response indicator may comprise a graphical element displayed on a view of an interactive space. For example, a response indicator may comprise a portion of the interactive space, a pop-up window, conversation bubble, and/or other graphical element.

By way of non-limiting illustration, the one or more colors may include one or more of a first color, a second color, a third color, and/or other colors. The first color may convey that a subject interaction is progressing toward attaining or accomplishing one or more objectives. For illustrative purposes, the first color may be green. The second color may convey that a subject interaction is falling behind attaining or accomplishing one or more objectives. For illustrative purposes, the second color may be red. The third color may convey that a subject interaction is stagnant with respect to attaining or accomplishing one or more objectives. For illustrative purposes, the third color may be yellow.

An explicit prompt may include suggestive, corrective, and/or encouraging text and/or other information. By way of non-limiting illustration, a prompt may comprise “might be heading down a wrong path, do you want to start over?” in response to subject interaction which is falling behind attaining or accomplishing one or more objectives. Another explicit prompt may be “What do you think the other person in this situation needs from you so you can accomplish your goal right now?”. Another explicit prompt may be “Is there a question you might ask?”. The above examples of prompts are for illustrative purposely only and not to be considered limiting.

In some implementations, quantitative feedback may be provided through presentation of one or more of a score, a rank, and/or other quantitative expressions. By way of non-limiting illustration, a score and/or rank may be based on one or more scales. By way of non-limiting illustration, a score between zero and 100 may be provided. A relatively higher score, e.g., in the range of seventy-five and 100, may be representative of subject interaction progressing toward attaining or accomplishing one or more objectives. A relatively mid-level score, e.g., in the range of forty-five to seventy-five, may be representative of subject interaction being stagnant. A relatively lower score, e.g., in the range of zero to forty-five, may be representative of subject interaction falling behind toward attaining or accomplishing one or more objectives. Ranking may be represented in a similar manner albeit on a numerical scale (e.g., 1-10) and/or grade scale (e.g., A-F).

The logging component 112 may be configured to generate behavior logging information reflecting the subject interaction with the interactive space based on one or more of the data collection information, the response indicators, and/or other information. Other information included by behavior logging information may include subject physical location (e.g., determined through GPS and/or other location sensor). In some implementations, generating the behavior logging information reflecting the subject interaction with the interactive space may comprise one or more of monitoring the subject interaction to identify portions of the subject interaction that satisfies the data collection information, generating individual timestamps associated with the identified portions of the subject interaction that satisfy the data collection information, and/or other operations.

Monitoring may include one or more of observing, checking, generating a text-based record, and/or other functionality. In some implementations, the monitored portions of the subject interaction may be associated with the individual timestamps to create a chronology of the subject interaction. Identifying portions of the subject interaction that satisfy the data collection information may include matching subject interaction with information specified by the data collection information. By way of non-limiting illustration, based on data collection information may specifying an identification of an individual objective, the interactive space and/or virtual content thereof defining the objective may be monitored. By way of non-limiting illustration, based on data collection information may specifying subject interaction with the interactive space toward attaining and/or accomplishing an individual objective, the subject interaction surrounding attaining and/or accomplishing the individual objective may be monitored. This may include monitoring one or more of control inputs by the subject, communications with one or more non-subject entities, a start time when attaining and/or accomplishing the individual objective, an end time after attaining and/or accomplishing the individual objective, whether attaining and/or accomplishing the individual objective was abandoned, and/or other features and/or functionality.

In some implementations, logging component 112 may be configured to generate aggregate behavior logging information and/or other information. The aggregate behavior logging information may include multiple iterations of behavior logging information generated over multiple iterations of the subject interaction with the interactive space. The logging component 112 may be configured to identify subject interaction patterns of the subject interaction from the aggregate behavior logging information. Subject interaction patterns may refer to when the same subject interaction occurs more than once when encountering the same or similar virtual content (e.g., a same or similar objective). By way of non-limiting illustration, a subject interaction pattern may include that the subject had abandoned a particular objective one more than one occasion. By way of non-limiting illustration, a subject interaction pattern may include that the subject has successfully attained and/or accomplished a particular objective on more than one occasion. The above descriptions of subject interaction pattern are for illustrative purposely only and are not to be considered limitation. Instead, it is to be understood that patterns may be expressed in other ways and/or may the identification of which may not be readily ascertainable by a therapist absent the aggregate behavior information.

In some implementations, logging component 112 may be configured to train a machine learning model and/or utilize artificial intelligence (AI) to identify subject interaction patterns of the subject interaction from the aggregate behavior logging information. The model may be trained based on one or more of input information, output information, and/or other information. The input information may comprise descriptions of subject interactions indicative of one or more pattern. The output information may comprise the one or more patterns. The machine learning model may include one or more of a neural network, a convolutional neural network, and/or other machine-learning framework. In some implementations, the machine learning model may be configured to optimize objective functions. In some implementations, optimizing objective functions may include one or both of maximizing a likelihood of the training set or minimizing a classification error on a held-out set.

The logging component 112 may be configured to analyze aggregate behavior logging information to identify patterns based on inputting aggregate behavior logging information into the trained model, AI, and/or other analysis component. The outputs of the trained model include one or more identified patterns. In some implementations, the model may continue to be trained (e.g., may learn) as the model is utilized. By way of non-limiting illustration, a successful output of the model in identifying one or more patterns may be provided as input into the model to further train the model.

The output component 114 may be configured to parse the behavior logging information into a structured data format. The therapist user may access the structured data format of the behavior logging information to assist the therapist with managing a treatment plan. The behavior logging information transformed into a structured data format may be stored a structured database. The therapist may search the database in order to analyze a subject's progress. The behavior logging information may be structured by virtue of the behavior logging information being represented by values of pre-defined, searchable, attributes. The attributes may be related to one or more of different ones of the data collection protocols from the data collection information, individual conditions, individual subjects, individual objectives, and/or other information. The therapist may input search queries into a user interface to search the database. The output may include behavior logging information which matches the queries. The output may be presented on the user interface in a manner which can be read and/or understood by the therapies. The output may include one or more of a table, a chart, and/or other information.

In some implementations, the information stored in the database may be stored in time sequence and/or stored by subject indefinitely to allow for later analysis as well as accessed dynamically in real time for feedback. In some implementations, the databases may be stored in a cloud server, while allowing the data to be tracked back to the individual subject, environment, and/or a group that the user was with. The database may be update automatically with each use. Permissions may be be set up so that individuals (e.g., therapists) can access data on subjects and/or researchers can access data on multiple subjects for research purposes.

It has been shown that therapy that tested subjects on multiple challenges, gives comfortable, but not harsh, feedback, and provides feedback relevant to their performance in the challenge, and in real time, reveals a pattern of behaviors and the subjects learned to accommodate the feedback in constructive ways reducing their errors. Prior to the features and/or functionality described herein, data may have contaminated in the virtual environment program itself and analysis may have been conducted manually. The system 100 addresses these downfalls by capturing the interaction and feedback in an automated database that also performs analytical pattern seeking analysis. Implementation of the system 100 has proved that access to factual data is much better than subject self-reported data. The database may record and provide factual data along with the analysis so the therapist has better data, especially behavioral trending data, upon which the therapist can structure the treatment program.

In some implementations, the output component 114 may be configured to generate an interaction report based on behavior logging information and/or other information. In some implementations, generating the interaction report based on the behavior logging information may comprise transcribing the identified portions of the subject interaction that satisfy the data collection information and/or other information. Transcribing may include describing the identified portions of the subject interaction in a human-readable format. The wherein the human-readable format may include descriptions of the identified portions of the subject interaction, a timeline, and/or other information. The interaction report may be presented in a user interface.

It is noted that while one or more features and/or functions described herein are directed to a single interaction with a virtual environment by a subject, this is for illustrative purposes only. Instead, it is to be understood that one or more features and/or functions described herein may be extended to treatment plans which may involve multiple iterations of subject access to a virtual environment. The virtual environment may change through one or more of the iterations. The virtual environment may consistently remain the same through one or more of the iterations. The virtual environment may change and/or remain the same based on subject interaction with one or more of the iterations. In some implementations, iterative application of a virtual environment in the subject therapy plan may facilitate “cognitive reorganization.” Cognitive reorganization through the system 100 may refer to the subjects use of the immersive learning environment provided by the system 100, where iterative challenges, feedback, and/or rehearsals may dis-equilibrate biases and/or constraints lacked in mental models not aligned with therapy goals in the adaptive unconscious. Subsequently there may exist opening for new knowledge, through iterative success and self-learning from feedback provided by the system 100. The system 100 may be used for constructing and habituating new mental models in the subjects, that pull the achieve to achieve the goal. The system 100 may achieve results which, historically, were only carried out face to face in exercises between therapist and subject.

The cognitive reorganization may be based on the iterative experience and immediate embedded feedback on the user's response to experiences. The feedback may be granular and/or low density (e.g., color-based response indicators), and/or may be provided by more detailed feedback (e.g., an explicit prompt). Having embedded feedback along a journey through a virtual environment along with explicit objectives have been shown to reorganize a user's default “theory” or interpretation of the situation, e.g., if the feedback is provided when the situation is being interpreted with an inappropriate or inaccurate model of the situation or scene and the user's actions indicate that. The subject may never actually be “instructed” but rather has to discover the correct interpretation of the situation through trial and error. Likewise, the subjects behavior is used as an indicator of his or her interpretation. Eventually, the result of subject interaction may be the development of a mental model of a situation that incorporates other's views, is less egoistic, and/or which takes into consideration all the information available and is generally more adaptive.

In some implementations, electronic storage 124 may store a repository of one or more of space information, therapy information, and/or other information. One or more components of processor(s) 126 may be configured to obtain information from the repository. Obtaining may include one or more of submitting queries, obtaining responses satisfying the queries, and/or other operations.

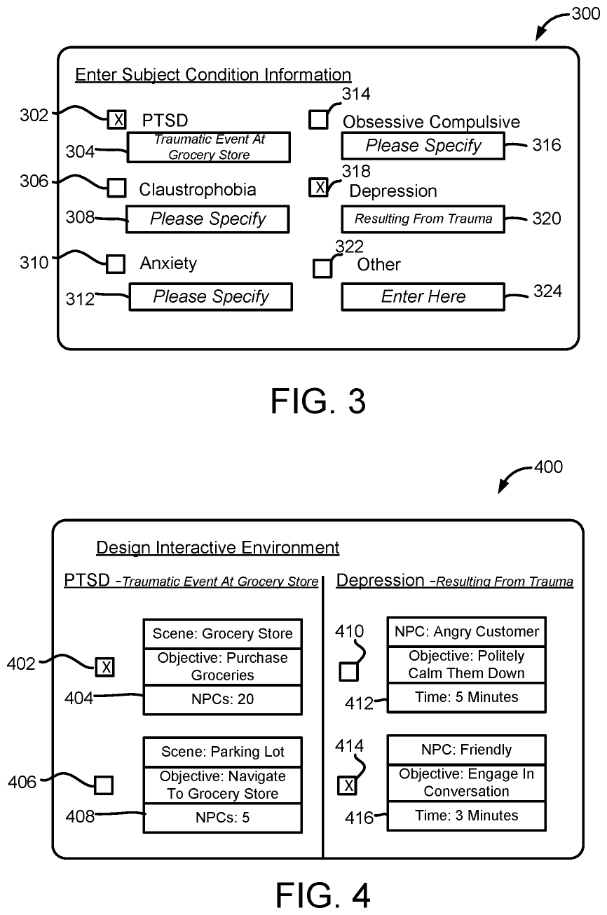

FIG. 3 illustrates an exemplary user interface 300 configured to obtain entry and/or selection of subject condition information. The user interface 300 may include one or more user interface elements configured to facilitate user interaction with the user interface 300. By way of non-limiting illustration, the user interface 300 may include one or more of a set of check boxes 302, 306, 310, 314, 318, and 322 configured to obtain user selection of individual mental distress conditions, a set of text input fields 304, 308, 312, 316, 320, and 324 configured to obtain user entry of additional description/specification for corresponding ones of the individual mental distress conditions, and/or other user interface elements. For illustrative purposes, the entry and/or selection shown in the figure is for “PTSD” which is further specified as a “traumatic event at a grocery store,” and “depression” which is further specified as “resulting from trauma.”

FIG. 4 illustrates an exemplary user interface 400 configured to obtain entry and/or selection of virtual content. The user interface 400 may be presented in response to the entry and/or selection of the subject condition information (e.g., FIG. 3) and may include individual sets of virtual content associated with the individual mental distress conditions. The user interface 400 may include one or more user interface elements configured to facilitate user interaction with the user interface 400. By way of non-limiting illustration, the user interface 400 may include one or more of a set of check boxes 402, 406, 410, and 414 configured to obtain user selection of individual sets of virtual content, a set of displays 404, 408, 412, and 416 showing representations (in this case, text descriptions) of corresponding ones of the individual sets of virtual content, and/or other user interface elements.

Individual sets of virtual content may be associated with the individual mental distress conditions. By way of non-limiting illustration, a first set 404 and a second set 408 may be associated with the mental distress condition “PTSD” and/or “traumatic event at a grocery store”; and a third set 412 and a fourth set 416 may be associated with the mental distress condition “depression” and/or “resulting from trauma.” Individual sets may include one or more of one or more scenes, one or more objectives, one or more virtual objects including NPCs, and/or other content. By way of non-limiting illustration, the first set may include a grocery store scene, an objective of purchasing groceries, an inclusion of twenty NPCs, and/or other virtual content. The second set 408 may include a scene of a parking lot, an objective of navigating to the grocery store, an inclusion of five NPCs, and/or other virtual content. The third set 412 may include an NPC having an angry demeanor, an objective of politely calming them down, an objective of doing so in a time limit of five minutes, and/or other virtual content. The fourth set 412 may include an NPC having a friendly demeanor, an objective of engaging in conversation with the NPC, an objective of doing so in three minutes, and/or other virtual content. For illustrative purposes, selections are shown as including the first set 404 via checking box 402 and the fourth set 416 via checking box 414.

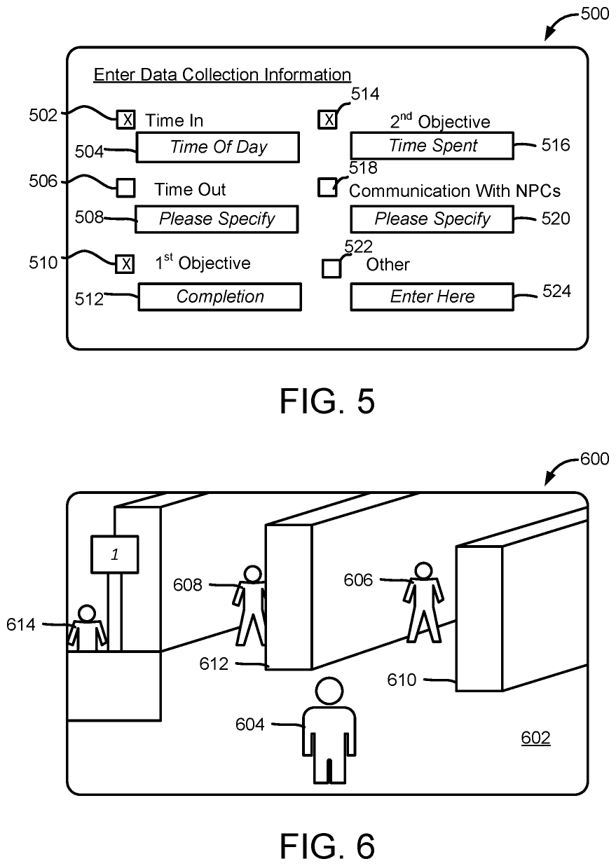

FIG. 5 illustrates an exemplary user interface 500 configured to obtain entry and/or selection of data collection information. The user interface 500 may include one or more user interface elements configured to facilitate user interaction with the user interface 300. By way of non-limiting illustration, the user interface 500 may include one or more of a set of check boxes 502, 506, 510, 514, 518, and 522 configured to obtain user selection of information to be collected, a set of text input fields 504, 508, 512, 516, 520, and 524 configured to obtain user entry of additional description/specification for corresponding ones of information to be collected, and/or other user interface elements. For illustrative purposes, box 502 may include “time in” (e.g., time the subject entered the interactive space), box 506 may include “time out” (e.g., time the subject exited the interactive space), box 510 may include “1^{st} objective” (e.g., collecting interaction related to accomplishing a first objective), box 514 may include “2^{nd} objective” (e.g., collecting interaction related to accomplishing a second objective), box 518 may include “communications with NPCs” (communications to and/or from NPCs), and box 522 may be for inputting other data collection information. For illustrative purposes, the selections are shown as “time in” which is further specified to collect what “time of day” the subject enters the interactive space, “1^{st} objective” which is further specified to collect whether the objective was completed, “2^{nd} objective” which is further specified to collect the amount of “time spent” on this objective.

FIG. 6 illustrates an exemplary user interface 600 showing an interactive space 602. A subject may interact with the interactive space by controlling a subject-controlled virtual entity 604. In the current depiction, the view of the interactive space 602 may be from a third-person perspective of the virtual entity 604. The interactive space 602 may include virtual content of a grocery scene including one or more NPCs and/or one or more objectives (see, e.g., FIG. 4). By way of non-limiting illustration, the virtual content may include one or more of a grocery shelves 610 and 612, one or more NPCs representing other customers 606 and 605, a teller 614 at a checkout counter, and/or other virtual content. The subject may interact with the interactive space to attain or accomplish the one or more objectives (see, e.g., FIG. 5) including one or more of control of the subject-controlled virtual entity 604 traversing through a topography of the interactive space, interaction with one or more virtual items, interaction with one or more NPCs 606, 608, and/or 614, and/or other interactions.

It is noted that the depictions in FIGS. 3-6 are for illustrative purposes and are not to be considered limiting. Instead, it is to be appreciated that user interfaces described herein may be arranged in different manners, include other virtual content, and/or may be expressed in other was in accordance with this disclosure.

Returning to FIG. 1, in some implementations, server(s) 102, client computing platform(s) 104, and/or external resources 122 may be operatively linked via one or more electronic communication links. For example, such electronic communication links may be established, at least in part, via network(s) 103 such as the Internet and/or other networks. Accordingly, the system 100 and/or components may be considered as part of the Internet-of-things. It will be appreciated that this is not intended to be limiting, and that the scope of this disclosure includes implementations in which server(s) 102, client computing platform(s) 104, and/or external resources 122 may be operatively linked via some other communication media.

An individual client computing platform of one or more client computing platforms 104 may include one or more processors configured to execute computer program components. The computer program components may be configured to enable a user associated with the individual client computing platform to interface with system 100 and/or external resources 122, and/or provide other functionality attributed herein to client computing platform(s) 104. By way of non-limiting example, the individual client computing platform may include one or more of a desktop computer, a laptop computer, a handheld computer, a tablet computing platform, a NetBook, a Smartphone, a gaming console, and/or other computing platforms.

External resources 122 may include sources of information outside of system 100, sources of space information and/or other information, external entities participating with system 100, and/or other resources. In some implementations, some or all of the functionality attributed herein to external resources 122 may be provided by resources included in system 100.

Server(s) 102 may include electronic storage 124, one or more processors 126, and/or other components. Server(s) 102 may include communication lines, or ports to enable the exchange of information with network(s) 103 and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 124 may comprise non-transitory storage media that electronically stores information. The electronic storage media of electronic storage 124 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 124 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 124 may include one or more virtual storage resources (e.g., cloud storage, a virtual private network, and/or other virtual storage resources). Electronic storage 124 may store software algorithms, information determined by processor(s) 126, information received from server(s) 102, information received from client computing platform(s) 104, and/or other information that enables server(s) 102 to function as described herein.

Processor(s) 126 may be configured to provide information processing capabilities in server(s) 102. As such, processor(s) 126 may include one or more of a physical processor, a digital processor, an analog processor, a digital circuit designed to process information, an analog circuit designed to process information, a state machine, and/or other mechanisms for electronically processing information. Although processor(s) 126 is shown in FIG. 1 as a single entity, this is for illustrative purposes only. In some implementations, processor(s) 126 may include a plurality of processing units. These processing units may be physically located within the same device, or processor(s) 126 may represent processing functionality of a plurality of devices operating in coordination. Processor(s) 126 may be configured to execute components 108, 110, 112, and/or 114, and/or other components. Processor(s) 126 may be configured to execute components 108, 110, 112, and/or 114, and/or other components by software; hardware; firmware; some combination of software, hardware, and/or firmware; and/or other mechanisms for configuring processing capabilities on processor(s) 126. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 126 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 126 may be configured to execute one or more additional components that may perform some or all of the functionality attributed below to one of components 108, 110, 112, and/or 114.

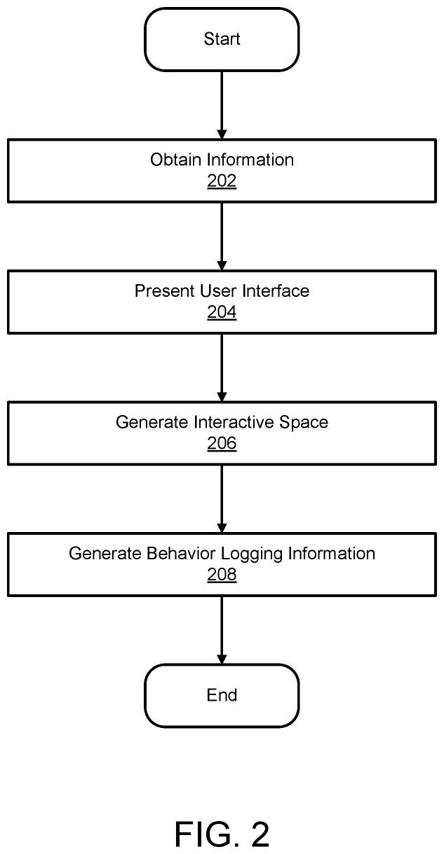

FIG. 2 illustrates a method 200 to provide mental distress therapy through subject interaction with an interactive space. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in a system comprising one or more processing devices (e.g., a digital processor, an analog processor, a digital circuit designed to process information, an analog circuit designed to process information, a state machine, and/or other mechanisms for electronically processing information), storage media storing machine-readable instructions, one or more physical objects, and/or other components. The one or more processing devices may include one or more devices executing some or all of the operations of method 200 in response to instructions stored electronically on electronic storage media. The one or more processing devices may include one or more devices configured through hardware, firmware, and/or software to be specifically designed for execution of one or more of the operations of method 200.

At an operation 202, one or more of space information, therapy information, and/or other information may be obtained. The space information may define virtual content of an interactive space. The therapy information may specify associations between the virtual content and one or more mental distress conditions. By way of non-limiting illustration, the space information may define first virtual content and/or other virtual content. The therapy information may specify an association between the first virtual content and a first mental distress condition, and/or other associations. In some implementations, operation 202 may be performed by one or more physical processors executing a component the same as or similar to user interface component 108 and/or space component 110 (shown in FIG. 1 and described herein).

At an operation 204, presentation of a user interface on a computing platform associated with a therapist user may be effectuated. The user interface being configured to obtain entry and/or selection of one or more of subject condition information, data collection information, and/or other information. The subject condition information may include individual mental distress conditions of a subject of the mental distress therapy, and/or other information. The data collection information may include information to be collected about subject interaction with the interactive space during the mental distress therapy. In some implementations, operation 204 may be performed by one or more physical processors executing a component the same as or similar to user interface component 108 (shown in FIG. 1 and described herein).

At an operation 206, interactive space including the virtual content associated with entered and/or selected ones of the individual mental distress conditions of the subject may be generated. The subject interaction with the interactive space may cause the subject to encounter the virtual content associated with the entered and/or selected ones of the individual mental distress conditions. By way of non-limiting illustration, the interactive space may be generated to include the first virtual content based on an entry and/or selection of the first mental distress condition. In some implementations, operation 206 may be performed by one or more physical processors executing a component the same as or similar to space component 110 (shown in FIG. 1 and described herein).

At an operation 208, behavior logging information reflecting the subject interaction with the interactive space based on the data collection information and/or other information. In some implementations, operation 208 may be performed by one or more physical processors executing a component the same as or similar to logging component 114 (shown in FIG. 1 and described herein).

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to provide a computer-generated interactive virtual space, the system comprising:
non-transitory electronic storage storing space information and therapy information, the space information defining virtual content of the interactive virtual space, the therapy information specifying associations between the virtual content and one or more mental distress conditions, wherein the virtual content includes one or more objectives that subject interaction with the interactive virtual space is intended to attain or accomplish, such that the space information defines first virtual content, and the therapy information specifies an association between the first virtual content and a first mental distress condition;
one or more physical processors configured by machine-readable instruction to:
effectuate presentation of a user interface on a computing platform associated with a therapist user, the user interface being configured to obtain selection of subject condition information and data collection information, the subject condition information including individual mental distress conditions of a subject of mental distress therapy, the data collection information including data to be collected about the subject interaction with the interactive virtual space during the mental distress therapy, wherein the subject interaction with the interactive virtual space to attain or accomplish the one or more objectives includes control of a direction of travel of a virtual avatar of the subject through a topography of the interactive virtual space and control of interactions of the virtual avatar of the subject with one or more virtual items and one or more non-user controlled virtual entities;
generate, at a server remote from the computing platform, the interactive virtual space including the virtual content associated with selected ones of the individual mental distress conditions of the subject, such that the interactive virtual space is generated to include the first virtual content based on a selection of the first mental distress condition;
effectuate presentation of the interactive virtual space on a mobile computing platform associated with the subject by communicating information defining the interactive virtual space from the server to the mobile computing platform, wherein the mobile computing platform is remote from server such that the mobile computing platform and server communicate via a wireless communication network;
obtain information conveying input by the subject into the mobile computing platform, the input including commands to control the direction of travel of the virtual avatar of the subject through the topography of the interactive virtual space and commands to control the interactions of the virtual avatar of the subject with the one or more virtual items and the one or more non-user controlled virtual entities; and
generate, at the server, behavior logging information reflecting the subject interaction with the interactive virtual space based on the data collection information.

2. (canceled)

3. (canceled)

4. The system of claim 1, wherein the user interface configured to obtain the selection of the subject condition information and the data collection information comprises a set of user interface elements, wherein the selection of an individual user interface element in the set of user interface elements causes a corresponding individual datum of the data to be collected during the mental distress therapy, wherein the data to be collected includes a start time of the subject interaction, an end time of the subject interaction, identification of the one or more objectives, and the subject interaction with the interactive virtual space toward attaining or accomplishing the identified ones of the one or more objectives.

5. The system of claim 4, wherein generating, at the server, the behavior logging information reflecting the subject interaction with the interactive virtual space based on the data collection information comprises:
monitoring the subject interaction to identify portions of the subject interaction that satisfies the data collection information; and
generating individual timestamps associated with identified ones of the portions of the subject interaction that satisfy the data collection information.

6. The system of claim 5, wherein the one or more physical processors are further configured by the machine-readable instructions to:
generate an interaction report based on the behavior logging information by:
transcribing the identified ones of the portions of the subject interaction that satisfy the data collection information into a human-readable format, wherein the human-readable format includes descriptions of the identified ones of the portions of the subject interaction that satisfy the data collection information.

7. The system of claim 1, wherein the one or more physical processors are further configured by the machine-readable instructions to:
effectuate presentation, on the user interface and in response to the selection of the subject condition information, of representations of individual sets of virtual content associated with the individual mental distress conditions, wherein the representations of individual virtual content include text descriptions of scenes, objectives, and virtual objects included in the individual virtual content arranged in a grid on the user interface, wherein the representations of the individual virtual content in the individual sets of virtual content are configured to be selected for inclusion in the interactive virtual space, such that responsive to the selection of the first mental distress condition, presentation is effectuated of representations of a first set of virtual content associated with the first mental distress condition, the first set of virtual content including the first virtual content.

8. The system of claim 1, wherein the one or more physical processors are further configured by the machine-readable instructions to:
effectuate presentation of response indicators in the interactive virtual space based on the subject interaction with the interactive virtual space, an individual response indicator conveying feedback of the subject interaction.

9. (canceled)

10. The system of claim 1, wherein the one or more physical processors are further configured by the machine-readable instructions to:
generate aggregate behavior logging information including the behavior logging information generated over multiple iterations of the subject interaction with the interactive virtual space; and
identify behavior patterns of the subject interaction from the aggregate behavior logging information.

11. A method to provide a computer-generated interactive virtual space, the method comprising:
obtaining space information and therapy information, the space information defining virtual content of the interactive virtual space, the therapy information specifying associations between the virtual content and one or more mental distress conditions, wherein the virtual content includes one or more objectives that subject interaction with the interactive virtual space is intended to attain or accomplish, such that the space information defines first virtual content, and the therapy information specifies an association between the first virtual content and a first mental distress condition;
effectuating presentation of a user interface on a computing platform associated with a therapist user, the user interface being configured to obtain selection of subject condition information and data collection information, the subject condition information including individual mental distress conditions of a subject of mental distress therapy, the data collection information including data to be collected about the subject interaction with the interactive virtual space during the mental distress therapy, wherein the subject interaction with the interactive virtual space to attain or accomplish the one or more objectives includes control of a direction of travel of a virtual avatar of the subject through a topography of the interactive virtual space and control of interactions of the virtual avatar of the subject with one or more virtual items and one or more non-user controlled virtual entities;
generating, at a server remote from the computing platform, the interactive virtual space including the virtual content associated with selected ones of the individual mental distress conditions of the subject, including generating the interactive virtual space to include the first virtual content based on a selection of the first mental distress condition;
effectuating presentation of the interactive virtual space on a mobile computing platform associated with the subject by communicating information defining the interactive virtual space from the server to the mobile computing platform, wherein the mobile computing platform is remote from server such that the mobile computing platform and server communicate via a wireless communication network;
obtaining information conveying input by the subject into the mobile computing platform, the input including commands to control the direction of travel of the virtual avatar of the subject through the topography of the interactive virtual space and commands to control the interactions of the virtual avatar of the subject with the one or more virtual items and the one or more non-user controlled virtual entities; and
generating, at the server, behavior logging information reflecting the subject interaction with the interactive virtual space based on the data collection information.

12. (canceled)

13. (canceled)

14. The method of claim 11, wherein the user interface configured to obtain the selection of the subject condition information and the data collection information comprises a set of user interface elements, wherein the selection of an individual user interface element in the set of user interface elements causes a corresponding individual datum of the data to be collected during the mental distress therapy, wherein the data to be collected includes a start time of the subject interaction, an end time of the subject interaction, identification of the one or more objectives, the subject interaction with the interactive virtual space toward attaining or accomplishing the identified ones of the one or more objectives.

15. The method of claim 14, wherein generating, at the server, the behavior logging information reflecting the subject interaction with the interactive virtual space based on the data collection information comprises:
monitoring the subject interaction to identify portions of the subject interaction that satisfies the data collection information; and
generating individual timestamps associated with identified ones of the portions of the subject interaction that satisfy the data collection information.

16. The method of claim 15, further comprising:
generating an interaction report based on the behavior logging information by transcribing the identified ones of the portions of the subject interaction that satisfy the data collection information into a human-readable format, wherein the human-readable format includes descriptions of the identified ones of the portions of the subject interaction that satisfy the data collection information.

17. The method of claim 11, further comprising:
effectuating presentation, on the user interface and in response to the selection of the subject condition information, of representations of individual sets of virtual content associated with the individual mental distress conditions, wherein the representations of individual virtual content include text descriptions of scenes, objectives, and virtual objects included in the individual virtual content arranged in a grid on the user interface, wherein the representations of the individual virtual content in the individual sets of virtual content are configured to be selected for inclusion in the interactive virtual space, including responsive to the selection of the first mental distress condition, effectuating presentation of representations of a first set of virtual content associated with the first mental distress condition, the first set of virtual content including the first virtual content.

18. The method of claim 11, further comprising:
effectuating presentation of response indicators in the interactive virtual space based on the subject interaction with the interactive virtual space, an individual response indicator conveying feedback of the subject interaction.

19. (canceled)

20. The method of claim 11, further comprising:
generating aggregate behavior logging information including the behavior logging information generated over multiple iterations of the subject interaction with the interactive virtual space; and
identifying behavior patterns of the subject interaction from the aggregate behavior logging information.
